(19) United States
(12) Patent Application Publication
(10) Pub. No.: US 2026/0026728 A1
(43) Pub. Date: Jan. 29, 2026

(54) MEDICAL DEVICE IMPLANTATION BY PERIOPERATIVE PATIENT EVALUATION PREPARATION

(71) Applicant: Medtronic, Inc., Minneapolis, MN (US)

(72) Inventors: Matthew J. Hoffman, St. Paul, MN (US); Ryan D. Wyszynski, Oak Grove, MN (US); Erin N. Hatlestad, Minneapolis, MN (US); Hyun J. Yoon, Vadnais Heights, MN (US)

(21) Appl. No.: 19/125,050

(22) PCT Filed: Oct. 16, 2023
(86) PCT No.: PCT/IB2023/060430

§ 371 Date: Apr. 28, 2025

Related U.S. Application Data
(60) Provisional application No. 63/381,439, filed on Oct. 28, 2022.

(51) Int. Cl. A61B 5/29; A61B 5/00; A61B 5/352

(52) U.S. Cl. CPC A61B 5/29; A61B 5/352; A61B 5/6847; A61B 5/7221; A61B 2562/0238

(57) ABSTRACT

This disclosure is directed to medical systems and techniques configured to test candidate implantation sites. An example medical tool includes a housing for one or more sensors. The housing is configured to be at least partially inserted into a body of a patient. The one or more sensors have a configuration corresponding to one or more sensors of a medical device to be implanted. The medical tool includes communication circuitry configured to transmit, to a computing device, sensor data generated from signals captured by the one or more sensors. The computing device is configured to run a test on the sensor data to identify an implantation site from at least one candidate implantation site for the medical device.

This application claims the benefit of U.S. Provisional Patent Application Ser. No. 63/381,439, filed Oct. 28, 2022, the entire content of which is incorporated herein by reference.

### FIELD

The disclosure relates generally to medical systems and, more particularly, medical systems configured for use in medical procedures implanting medical devices in patients.

### BACKGROUND

Medical systems may monitor various types of data of a patient or a group of patients for one or several purposes. Amongst the numerous examples, some medical systems may record measurements of a patient and electrical activity of their heart as indicia of cardiac health for that patient, which may be memorialized in raw data and/or processed data formats. For example, electric signals representing cardiac activity over a period of time may be memorialized as a cardiac electrogram (EGM), and then processed into other indicia of the cardiac health of the patient. In some examples, the medical system may monitor the cardiac EGM to detect one or more types of an arrhythmia, such as bradycardia, tachycardia, fibrillation, or asystole (e.g., caused by sinus pause or AV block).

Medical professionals may use such a medical system on their patients for a number of reasons, such as having the medical system record patient data for future use. Some medical professionals rely on that data to provide their patients with the best medical care. For various purposes, a medical professional may program the medical system to operate as desired, which may be in accordance with a certain algorithm and/or configurable settings, and calibrate medical system components to detect health events, deliver a therapy, and so forth. In some examples, the medical system may include one or more medical devices to collect various measurements used to detect changes in patient cardiac health. In some examples, the patient may undergo a procedure to insert or attach the medical device in or on the patient's body, respectively.

### SUMMARY

Medical systems and techniques as described herein facilitate medical device implantations and/or attachments in or at a location that may enhance the functionality of the medical device to the benefit of a patient who may be prone to acute health events. In general, these systems and techniques advantageously use tools, sensors, and other mechanisms to guide a clinician performing the implantation (including any support system) in successfully completing the implantation. As used herein, implantation, implant, or implanting, may include insertion, insert, or inserting, respectively.

A variety of medical devices (e.g., implantable devices, wearable devices, etc.) may be configured to monitor and collect data associated with patient physiology and detect changes in patient health that correlate to changes in data recording the patient physiology. A medical device may be designed to detect or predict (actual or potential) health events by achieving a certain performance level and normally would meet that expectation for the patient, but for its initial configuration. Such an initial configuration may include, but is not limited to, issues with the placement of the medical device within (or on) the patient's body. The present disclosure describes techniques which may be employed to ensure or increase the likelihood of a successful implantation at a preferred location and avoid or reduce a likelihood of an explantation and re-implantation, or a repositioning of the medical device once implanted.

The medical systems and techniques described herein disclose a medical tool that, in some examples, such as where the medical tool is used with an implantable device, may be configured for partial insertion into a body of the patient, including partial insertion at or relatively near a candidate implantation site for a medical device. The present disclosure provides examples of the use of sensor data to provide valuable insight for evaluating/testing the candidate implantation site (e.g., for the medical device or other devices). To illustrate by way of an example technique where the candidate implantation site refers to a location in or around a thoracic cavity of the body, a computing device may evaluate sensor data of the candidate implantation site using various criteria and determine whether the candidate implantation site is appropriate for the patient. As used herein sensor data may include sensor signals and/or physiological parameters or other parameters derived from such sensor signals.

For the above candidate implantation site, the medical tool may be manufactured to include a housing for one or more sensors and with a rigid body configured for partial insertion in or around a thoracic cavity of the body. Each sensor within or about the housing of the medical tool described herein may be configured to operate similarly to a corresponding sensor in the medical device to be implanted in the body of the patient. In this manner, signals generated by the sensor of the medical tool simulates (e.g., matches) signals that would be generated by the corresponding sensor of the medical device should the clinician insert the medical device into the candidate implantation site. To enable the above evaluation of the candidate implantation site, the medical tool is configured with communication circuitry to communicate, to an external computing device, any sensor data generated from signals of the one or more sensors of the medical tool. The computing device may be configured to (e.g., automatically) execute a test on the sensor data of the medical tool, in response to receiving the sensor data of the medical tool, and then, output, via an output device, content indicating test results. Example test results may indicate a validation of the candidate implantation site, an identification of a more appropriate implantation site, an indication that the candidate implantation site is not appropriate, a cancellation of the implantation procedure for a lack of an appropriate implantation site, or the like.

Testing one or more candidate implantation sites and determining which site(s) may be an appropriate implantation site(s) or which candidate implantation site may be more appropriate than other candidate implantation sites may not be practicably performable in the human mind. For example, a number of candidate implantation sites may be evaluated (e.g., tested for appropriateness of implantation) and compared to determine an appropriate (or, in some cases, a best) site for implantation. While a tool may be partially inserted into a patient to perform such test(s) at one or more candidate implantation sites, the patient may be administered anesthesia (local or general). If such a tool were to output sensed signals to a display for viewing and a clinician were to view such sensed signals to determine whether the sensed signals to determine the results of one or more tests for each candidate implantation site and evaluate which candidate implantation sites may be appropriate, more appropriate, or most appropriate for implantation of the implantable device, the time taken to conduct such evaluation may exceed the duration of the pain blocking effect of the anesthesia on the patient and may require further administration of anesthesia. Moreover, if multiple tests are performed on multiple sensed signals, the analysis may be too complex to be conducted in a human mind, particularly if multiple candidate implantation sites are involved.

While primarily described herein as a medical tool for use with an implantable device, it should be noted that a medical tool of this disclosure may be used to find an appropriate location or position for an external sensing device on the exterior (e.g., on the skin) of a patient. In such an example, the medical tool may or may not be configured to be partially inserted into a patient.

In view of the above, the present disclosure describes a technological improvement and/or a technical solution integrated into at least one practical application. The medical systems and techniques described herein may enhance medical device implantation procedures with informed guidance, thereby mitigating or eliminating altogether the problems associated with other approaches, such as when the clinician lacks information as to which locations on or in the patient's heart provide sufficient signals of electrical activity. Enabling the clinician to test and evaluate the medical device as an implant for a patient not only improves device capabilities, but also reduces a likelihood of faulty/suboptimal performance or even complete malfunction of an implanted device. As another benefit, certain devices required for conventional implantations may no longer be necessary.

In one example, a medical system comprises one or more sensors configured to sense patient activity; sensing circuitry configured to provide patient activity data based on the sensed patient activity; and processing circuitry configured to:

In another example, a method comprises:

In another example, a non-transitory computer-readable storage medium comprises program instructions that, when executed by processing circuitry of a medical system, cause the processing circuitry to:

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2 is a block diagram illustrating an example configuration of a medical device, in accordance with one or more examples of the present disclosure.

FIG. 3 is a block diagram illustrating an example configuration of the external device of FIG. 1, in accordance with one or more examples of the present disclosure.

FIG. 4 is a block diagram illustrating an example system that includes an access point, a network, external computing devices, such as a server, and one or more other computing devices, which may be coupled to the medical device and external device of FIGS. 1-4, in accordance with one or more examples of the present disclosure.

FIGS. 5A-5C are each an illustration of an example medical tool for use in an implantation of the medical device of FIGS. 1-2, in accordance with one or more examples of the present disclosure.

FIG. 6 is a flow diagram illustrating an example operation for evaluating a candidate implantation site, in accordance with one or more examples of the present disclosure.

Like reference characters denote like elements throughout the description and figures.

### DETAILED DESCRIPTION

In general, medical systems according to this disclosure implement techniques for medical device implantation. To support the medical systems and techniques described herein, the present disclosure describes a number of technologies of which one example may be a medical tool for use by clinicians that is configured to obtain feedback regarding implantation location quality as part of the preparation for an implantation procedure. Some medical systems employ the above medical tool to implement example techniques for delivering an implant (e.g., an implantable medical device (IMD)) to an implantation site of a patient's body.

According to some example techniques, a medical system including the medical tool may be configured to evaluate the implantation site for suitability such as when a clinician utilizes the medical tool to test one or different potential implantation locations. For example, the test may be run by a computing device that receives sensed data from the medical tool. The following description discloses a number of possible tests that the clinician could run, each configured to ensure or improve the likelihood that each medical implantation procedure correctly places that medical device in or on a patient's body such that the medical device is able to capture and store data recording accurate physiological and other patient information. Some medical systems and techniques process the feedback using different metrics for implantation location quality prior to insertion of the implant and/or the like. The present disclosure describes medical systems and techniques that, based on the processed feedback, may identify a best possible (or acceptable) implantation site for capturing a patient activity of interest (e.g., cardiac electrical activity) and direct the implantation procedure accordingly.

The present disclosure describes a number of benefits and advantages that can be attributed to using the medical tool of this disclosure for the medical device implantations. For instance, having a number of candidate implantation sites, some medical systems and techniques find the candidate implantation site by selecting an area on the patient's body that can be attributed with a highest accuracy and/or most insight into the patient's physiology and/or overall health. According to the techniques of this disclosure, clinicians may employ a medical tool as described herein instead of expensive and/or complicated equipment. A clinician can use the medical tool for implantations without having to admit the patient into a general hospital or a more exclusive medical facility that specializes in implantations. Therefore, having access to a medical tool described herein may enable the clinician to perform implantations at a significantly larger number of facilities.

Instead of a hospital, a certain type of medical facility, which may be referred to as an implantation clinic, may handle implantation procedures for patients. This type of medical facility may not have (e.g., proprietary) equipment capable of testing the medical device before, during, and/or after implantation. After safely implanting the implant, the patient and their clinician may test the implant at the same implantation clinic or a non-medical facility (e.g., a home of a remote monitoring patient) instead of a specialized facility capable of testing the implant, which may be known as a follow-up clinic for example, using a same computing device.

To enable testing at the implantation clinic without the testing equipment, medical personnel may use a medical tool as described herein. For one, the implantation clinics may not have access to a clinician programmer and could benefit from a perioperative tool to assess implant location quality. Using a medical tool of this disclosure at the implantation clinic may reduce or potentially eliminate the need for a clinician programmer during the implantation procedure. This reduction/elimination may encourage otherwise deterred clinicians to implant implantable medical devices (IMDs) such as insertable cardiac monitors (ICMs) at implantation clinics. After insertion of a portion of the medical tool into a patient, electrodes of the medical tool may sense electrical activity of a candidate implant site (e.g., an area within the body such as a thoracic cavity) and provide sensed data to determine the quality or effectiveness of the candidate implantation site for the implantation. The medical tool may further include, or form a portion of, an implanter configured to physically implant the implantable device. The implanter may be configured to evaluate the electrical activity (e.g., R-wave amplitude before, during, and/or after implantation).

An IMD may be configured with one or more electrodes for continuously measuring the patient's cardiac activities (e.g., electrical activities of the heart) by sensing a location near the patient heart and recording such measurements as patient data. Example IMDs that may collect patient data may include an ICM, a pacemaker/defibrillator, a ventricular assist device (VAD), or the like.

Consider, for instance, an example medical system where the implant is an ICM to be inserted proximate to, onto, or into the patient's heart, criteria may be defined to represent a level of accuracy at which the cardiac monitor operates effectively. If a sensor (e.g., an electrode) were to be positioned at a location of the insertion, the level of accuracy refers to a quality measure of any sensed electrical activity by that sensor. If the clinician tests a first candidate implantation site and determines that electrical activity can be sensed at a level of accuracy that fails to meet a threshold (as an example criterion), a medical system including the medical tool, may determine that the first candidate implantation site fails to satisfy the criterion. In some examples, the medical system may suggest a second candidate implantation site as discussed further herein. If the clinician tests a second candidate implantation site (either suggested by the medical system or determined by the clinician), the medical system may determine that the second candidate implantation site satisfies the criterion (e.g., meets the threshold), in which case the clinician may decide to implant the implantable device at the second candidate implantation site. Alternatively, the clinician may use the medical tool, or a system including the medical tool, to evaluate at least one third candidate implantation site and possibly, identify a more suitable location than the second implantation site for selection, for example, if having the medical tool at the selected third implantation site provides the most accurate electrical activity data for health event monitoring/detection analysis, or a higher level of accuracy than the second candidate implantation site.

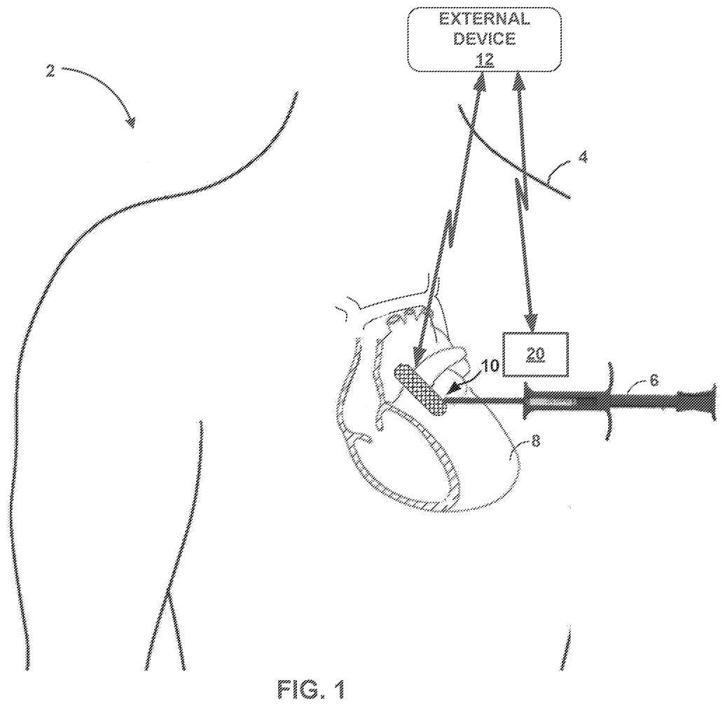

FIG. 1 illustrates the environment of an example medical system 2 in conjunction with patient 4, in accordance with one or more techniques of this disclosure. The example techniques may be used with a medical tool 6 (also referred to herein as “tool 6”), which may be communicably coupled with at least one of IMD 10, wearable device 20, external device 12, and/or other devices not pictured in FIG. 1. It should be noted that items depicted in FIG. 1 may not be shown to scale. For example, tool 6 may include a cavity for receiving IMD 10 and be configured to implant IMD 10 in patient 4, in which case IMD 10 may be small enough to fit within the cavity of tool 6.

Tool 6 is an electro-mechanical device and is described herein as being beneficial to clinicians who desire to implant medical devices, such as IMD 10, into their patients, which may result in those patients having more implants that operate more effectively. Tool 6 may alternatively, or additionally, be used by clinicians who desire to position external monitoring devices, such as wearable device 20, upon their patients, which may result in those patients having more effective external monitoring devices.

IMD 10, which may be in wireless communication with at least one of external device 12 and/or other devices not pictured in FIG. 1, represents one example medical device to be inserted into patient 4 by a clinician. In some examples, IMD 10 is implanted outside of a thoracic cavity of patient 4 (e.g., subcutaneously in the pectoral location illustrated in FIG. 1). IMD 10 may be positioned near the sternum or just below the level of the heart of patient 4, e.g., at least partially within the cardiac silhouette. IMD 10 includes a plurality of electrodes (not shown in FIG. 1) and is configured to sense a cardiac EGM via the plurality of electrodes. In some examples, IMD 10 takes the form of the LINQ™ ICM or LINQ II™ available from Medtronic, plc. Wearable device 20 may similarly be in wireless communication with at least one of external device 12 and/or other devices not pictured in FIG. 1. Wearable device 20 is shown as a patch, but may take other forms, such as a watch, a necklace, or the like. The discussion hereinafter is more directed to IMD 10 than to wearable device 20 for simplicity purposes. It should be noted that wearable device 20 may operate similarly to IMD 10 except that wearable device 20 may sense EGM signals external to patient 4.

External device 12 may be a computing device with a display viewable by the user and an interface for receiving user input to external device 12. In some examples, external device 12 may be a general purpose device, such as a smartphone, a tablet computer, a notebook computer, a desktop computer, a workstation, one or more servers, or another computing device that may run an application that enables the computing device to interact with IMD 10 and/or tool 6. In some examples, external device 12 may be a special purpose device, specifically designed to communicate with tool 6 and IMD 10.

External device 12 is configured to communicate with tool 6, IMD 10 and, optionally, another computing device (not illustrated in FIG. 1), via wireless and/or wired communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies). As explained in detail further below, external device 12 may also be, or alternatively be, configured to communicate with tool 6 via wired communication.

External device 12 may be used to configure device settings and/or operational parameters for IMD 10. External device 12 may be a general purpose device or a special purpose device for enabling clinician control over IMD 10, for example, by modifying various ones of such settings and/or parameters as directed by the clinician (e.g., a programmer) to control performance of IMD 10. Because these settings/parameters affect IMD 10 operation in general and for specific functionality (e.g., asystole detection, heart failure detection, and/or the like), calibrating their values allows the clinician to predict how IMD 10 may benefit patient 4's health and/or the monitoring of patient 4's health.

External device 12 may be used to retrieve data from tool 6 and/or IMD 10. The retrieved data may include values of sensor data including various physiological measurements and (possibly) indications of episodes of arrhythmia or other maladies. The sensor data includes sensor signals (e.g., raw sensor data) and/or physiological parameters (e.g., processed sensor data). The sensor signals included in the sensor data may be indicative of electrical activity of the heart of patient 4. External device 12 may retrieve cardiac EGM segments from tool 6 and/or IMD 10, for instance, as a monitoring device. External device 12 may retrieve other sensor signals and/or physiological parameters from tool 6 and/or IMD 10, such as optical sensor signals, accelerometer signals, impedance signals, pressure sensor signals, temperature sensor signals, or other types of sensor signals, and/or physiological parameters derived from such signals.

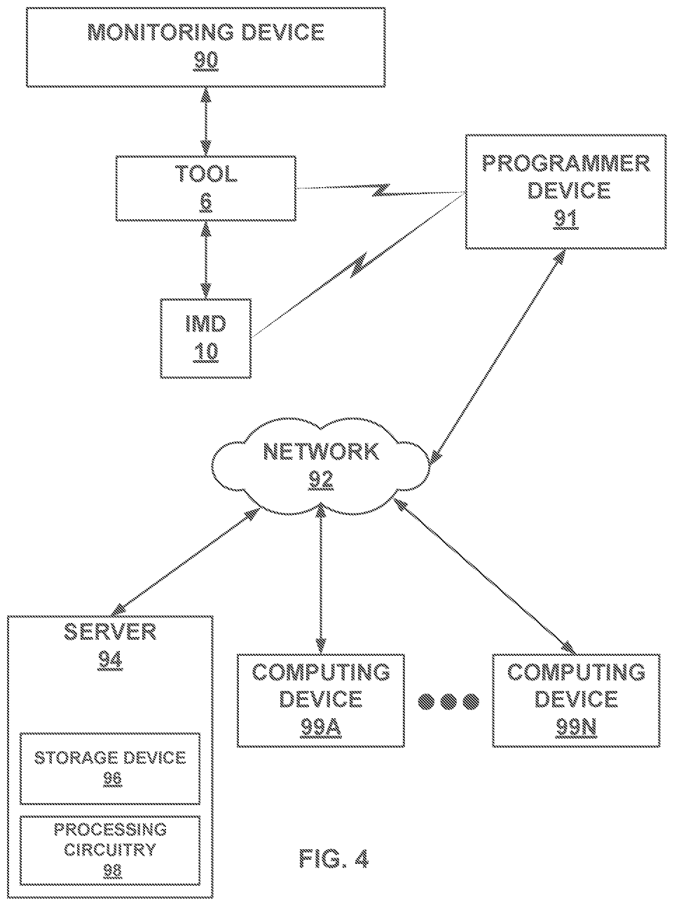

In some examples, external device 12 may be configured to perform specific functionality, such as a programmer for IMD 10. As explained in detail in the description of FIG. 4, medical system 2 may involve the programmer calibrating the settings/parameters being programmed in IMD 10 based on sensor data received from tool 6. In some examples, this specific functionality may be implemented in circuitry of external device 12. In some examples, this specific functionality may be implemented in one or more applications that may be executed by processing circuitry of external device 12 to provide such functionality.

Tool 6 refers to any mechanical/electrical device as set forth herein. In general, clinicians may use tool 6 to successfully delivery medical devices, such as IMD 10, as implants into patient 4, for example, by providing the clinicians with insight into a location within patient 4 as a candidate implantation site. Further, tool 6 may allow the clinicians to test alternate locations and ultimately find a more appropriate implantation site than the candidate implantation site. Additionally, or alternatively, clinicians may use tool 6 to successfully position external devices for attachment to patient 4.

Communication circuitry (e.g., communication circuitry) and/or other circuitry including sensing circuitry within tool 6 may be directed to transmit various sensor data. Sensor(s) including electrode(s) may be formed or placed on an outer surface of tool 6. Circuitry may be inserted or placed on an inner aperture of tool 6. In another alternative example, an antenna may be formed or placed on, or within, the rigid body of tool 6. In some examples, an insulative material may form the housing to enclose the antenna (if present) and/or the communication circuitry, for example, from fluids such as body fluids.

Processing circuitry of medical system 2, e.g., of one or more of IMD 10, external device 12, one or more other computing devices which are not shown in FIG. 1, or any combination thereof, may be configured to perform the techniques described herein. The processing circuitry of medical system 2 may employ various known mechanisms to capture (e.g., sample) various sensor data over a period of time and then, use that sensor data to evaluate the location as a candidate implantation site for IMD 10. Electrodes within or about tool 6 may be configured to sense electrical activity of a candidate implant site (e.g., a location or area in the body, such as heart 8) and provide sensed data to determine implant location quality, for example, in terms of accuracy at some point-in-time before, during, and/or after an implantation into a patient. By comparing the sensed data with expected or desired measurements or signals (e.g., expected or desired cardiac measurements or signals), a predicted accuracy of the implant's sensing configuration may be determined or evaluated. Examples of the sensed data include, but not limited to, the following: R-wave amplitude data; impedance; Bluetooth Low Energy (BLE) signal strength; heart sound volume (e.g., amplitude of a sensed heart sound), respiration sound volume (e.g., amplitude of a sensed respiration sound signal), among others.

In some examples, a number of algorithms may predict a risk of poor implant positioning based on the sensed data. As such, tool 6 may perform one or more tests to determine the appropriateness of a candidate implantation site. For example, a candidate implantation site may be appropriate if signals sensed by one or more sensors of tool 6 at the candidate implantation site have sufficient characteristics (e.g., as compared to threshold(s)) to pass the one or more tests. Such characteristics may include amplitude, signal strength, signal-to-noise ratio, or the like. A candidate implantation site that is not associated with sufficient characteristics may not be appropriate for implantation (e.g., may fail the test(s)) or may be less appropriate for implantation than another candidate site that is associated with more sufficient characteristics, or whose associated characteristics are deemed better (e.g., higher amplitude, higher signal strength, higher signal-to-noise ratio, or the like) than candidate implantation site.

For example, a sensed R-wave amplitude that does not meet a predetermined R-wave threshold (e.g., is lower than an R-wave threshold) may result in more false-positive and/or false-negative identifications of arrhythmia by IMD 10 and/or external device 12 than a sensed RT-wave amplitude that does meet the R-wave threshold. For example, a BLE signal whose signal strength does not meet a predetermined signal strength threshold (e.g., is lower than the signal strength threshold) may result in more connection drop-outs and a shorter battery charge and/or life due to repeated transmission attempts to transmit the same data than a BLE signal whose signal strength does meet the signal strength threshold. A candidate implant location whose sensed data does not meet such thresholds may have a higher risk of being a poor implant location than a candidate implant location whose sensed data does meet such thresholds. In some examples, a candidate implant location whose sensed data meets less thresholds than another candidate location may have a higher risk of being a poor implant location than the other candidate implant location. In some examples, at least one of such algorithms may be a machine learning or artificial intelligence algorithm which may be trained on pre-implantation, intra-implantation, and post-implantation sensor data (which may include sensor data from a plurality of tools 6 and/or IMDs 10). Such an algorithm may predict a risk of issues arising with IMD 10 due to implantation at a given candidate implantation site, based on sensed data from tool 6.

The following describes different examples of tool 6. Some examples of tool 6 may be configurable to transmit various sensor data to at least one external computing device (e.g., external device 12) by a variety of mechanisms. One mechanism involves a physical connection between tool 6's electrodes and IMD 10's electrodes and communication circuitry (e.g., wireless telemetry of IMD 10) for transmitting sensor data, such as R-wave amplitude data, to external device 12. A second mechanism involves components (e.g., ports) that are exposed on a handle of tool 6 and connected (by wire) to external device 12, for example, in the event the sensor data is cardiac electrogram (EGM) data, via cardiac EGM cables. A third mechanism involves separate communication circuitry, such as BLE communication circuitry incorporated into tool 6 that transmits, via telemetry, the sensor data to external device 12.

One example tool 6 described herein may be configured to capture various signals of patient information (e.g., physiological parameters of patient 4) from a certain location on the patient's body. Tool 6 and/or external device 12 may evaluate the various signals and any (e.g., embedded) patient information for satisfaction of one or more implant quality criterion. An example criterion may be a threshold (e.g., minimum/maximum) quantity or quality which may be based on, or indicative of, a particular signal quality (e.g., a preferred signal quality). An example technique described herein may set the threshold such that if the medical device (e.g., IMD 10) obtains that signal quality when implanted at the same location, the medical device is likely to work properly and, patient 4 is most likely to benefit from a properly working implant. As used herein, signal quality may include a signal strength (which may include an amplitude), a signal-to-noise ratio, a noise strength in the signal, or the like.

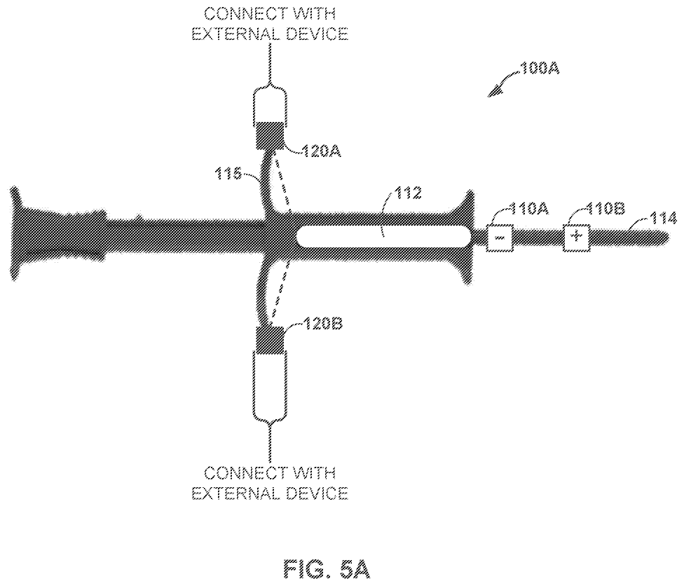

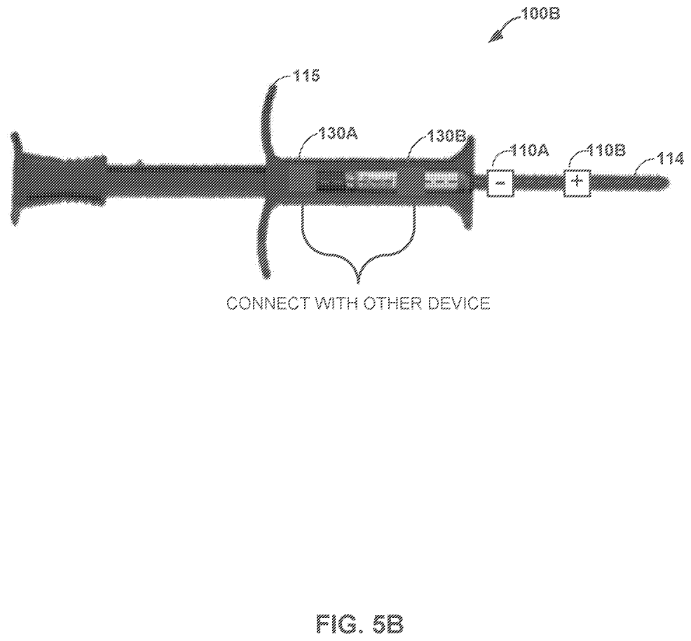

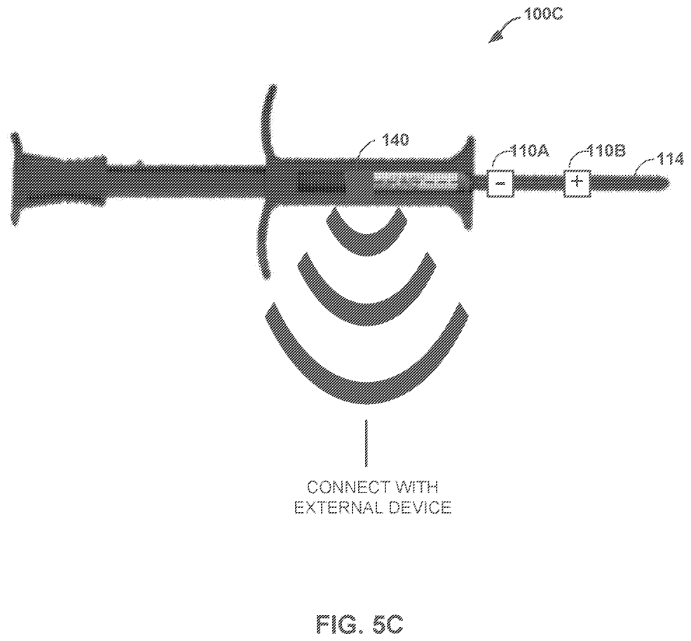

During implementation of IMD 10, or after the clinician successfully completes the implantation of IMD 10 into patient 4, the clinician may continue to use tool 6 to sense data via the one or more sensors of tool 6. As described in detail below for FIG. 5B, which depicts an example of tool 6, electro-mechanical components known as compression contacts may communicably couple with corresponding electrodes of IMD 10, and via communication circuitry of IMD 10, may transmit the sensor data to external device 12 for evaluation. In this manner, the implantation site may be tested for fitness or appropriateness during insertion of IMD 10, or even after IMD 10 is inserted into patient 4. In some examples, any intra-implementation or post-implantation test(s) may be combined with any test performed on the sensor data provided prior to implanting IMD 10. For example, external device 12 may compare the pre-implantation test results with the intra-implantation and/or post-implantation test results to determine whether the implantation of IMD 10 may be considered successful and/or to provide training data for a machine learning or artificial intelligence algorithm.

Once successfully implanted, IMD 10 may commence (e.g., regular) transmissions of various datasets (of patient information) to external device 12, and by comparing one or more of those datasets to the sensor data provided by tool 6, external device 12 may determine a post-implant quality. For example, a software application may run on external device 12 to evaluate those datasets in terms of different metrics (e.g., accuracy) and determine if patient 4 would benefit from having IMD 10 repositioned, for example, at a different implantation angle and/or at a different implantation site. A computing device operated by the clinician (e.g., external device 12), may perform one or more tests of the implantation site, and/or other computing devices, including other examples of external device 12, such as an ECG monitor and/or a programmer for IMD 10, may be utilized to evaluate implant quality.

In another example, an example of tool 6 may be combined with one or more devices, forming a separate example of tool 6. For instance, tool 6 may include or be incorporated into an implanting device (e.g., an implanter). The implanter may be configured to implant IMD 10 into patient 4.

Tool 6 and IMD 10 may be configured to record the same sensor data, different sensor data, or some combination of each. Given that IMD 10 is designed for patient health monitoring (in general) and cardiac arrhythmia detection (specifically), tool 6 may be configured to collect sensor data for use in same or different health event detection functionality. In this regard, tool 6 may be used to test the efficacy of one or more proposed locations or areas within patient 4 to implant IMD 10. The sensor data provided by tool 6 may correspond to the device settings and/or operational parameters for IMD 10. External device 12 may leverage the sensor data to control performance of IMD 10 and, for example, the patient monitoring/arrhythmia detection functionality of IMD 10. It should be noted that the above settings/parameters may be used in a number of algorithms that could be implemented by IMD 10 for detecting an occurrence of an arrhythmia in time-stamped patient information (e.g., a cardiac EGM segment) and thus, may be calibrated to direct the algorithm's execution in any manner desired by a clinician. For instance, the clinician may use external device 12 to execute logic in software/hardware configured to map the sensor data provided by tool 6 to a set of values for the settings/parameters that are known, or suspected to be most effective or (at least) more effective than default settings/parameters.

In other examples of medical system 2, the processing circuitry of medical system 2 in may execute same or similar logic for wearable device 20 to be attached to patient 4 as the logic executed by the processing circuitry for IMD 10 and/or tool 6. In this manner, wearable device 20 may be successfully positioned and attached to patient 4 by some or all of the techniques described herein in the same manner described herein with respect to IMD 10 and/or tool 6.

In this manner, the medical systems and techniques of this disclosure may advantageously enable improved performance by medical devices in their designated functionality, such as in the detection of changes in patient health and, consequently, better evaluation of the condition of patient 4.

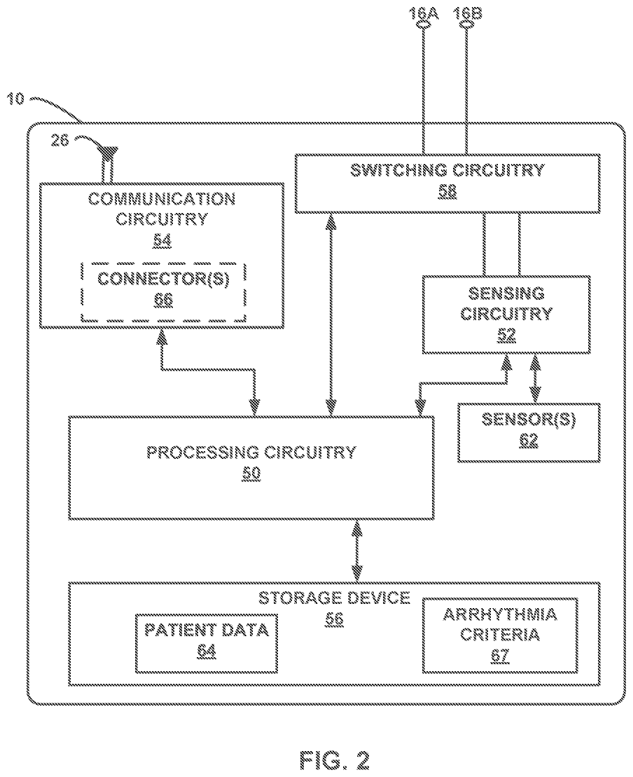

FIG. 2 is a functional block diagram illustrating an example configuration of IMD 10 of FIG. 1 in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to sense electrical activity of the heart of patient 4, for example by selecting electrodes 16 and polarity, referred to as the sensing vector, used to sense the cardiac electrical activity, as controlled by processing circuitry 50. Sensing circuitry 52 may sense signals from electrodes 16, e.g., to produce cardiac EGM data or ECG data as examples of the patient data. In some examples, sensing circuitry 52 may configure electrodes 16 to sense an impedance within the body of patient 4.

Sensing circuitry 52 may monitor signals from sensors 62 to process various sensor measurements to include as part of the patient data. Examples of one or more sensors 62 configured to sense patient data include an accelerometer (e.g., a three-axis accelerometer), a pressure sensor, an optical sensor, a gyroscope, a temperature sensor, and/or the like. Various metrics enable standardized measurement(s) for each sample (e.g., timestamp) of the sensed patient data and differentiation between multiple samples. In some examples, sensing circuitry 52 may include one or more filters and amplifiers for filtering and amplifying signals received from electrodes 16 and/or sensors 62.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes. In some examples, communication circuitry 54 may include one or more connector(s) 66. Connector(s) 66 may be configured to connect with connectors of tool 6 such that sensor data collected by tool 6 may be input to IMD 10 and may be transmitted by communication circuitry 54 to external device 12 as one example of a manner in which sensor data from tool 6 may reach external device 12. For example, connector(s) 66 may be configured to physically and directly connect to corresponding one or more connector(s) of tool 6 which may be coupled to sensing circuitry or electrodes of tool 6.

Connector(s) 66 may be any mechanical-electrical component capable of providing an electrical contact for reliable connectivity/communication; some examples of connector(s) 66 include mechanical-electrical components knowns as compression connectors or compression contacts. In general, each of connector(s) 66 is composed of a conductive material (e.g., a wire) and is configured to couple with one or more corresponding (e.g., compatible) interfaces of the above other device, including the medical device for the purposes of achieving a successful implantation.

Each of connector(s) 66 is manufactured with a periphery surface configured to establish a coupling with a compatible interface. One example of such an interface may include electrical-mechanical hardware through which each of connector(s) 66 may be mounted to a printed circuit board (PCB) or mated with logic circuitry on that board. Mounting blocks, as one example of the above electrical-mechanical hardware, may be coupled with compression connectors to form a compression connection.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more settings and/or operational parameters of IMD 10 and/or data collected by IMD 10 for transmission to another device using communication circuitry 54. Data stored by storage device 56 and transmitted by communication circuitry 54 to one or more other devices may include the patient data described herein for suspected changes in and/or indications of changes in patient health.

Sensing circuitry 52 may capture signals from electrodes 16 and/or any one or more of sensors 62, for example, to produce the patient data for processing by hardware/software (e.g., within IMD 10, external device 12, and/or another device), thereby facilitating (e.g., remote) monitoring and detecting changes in patient health (e.g., by an external computing system such as a network-accessible device running a computing service). The above-mentioned hardware/software may include software applications configured with logic to generate additional patient data, for example, data further describing patient health in terms of a health event history over a time period. In one example, processing circuitry 50, executing logic herein referred to as detection logic, applies various criteria to a timestamped sample of sensor measurements to determine if the sample is evidence of an arrhythmia likely to cause a change in patient health or, otherwise, negatively affect the patient's heart.

Sensing circuitry 52 may convert, to digital form, signals corresponding to the sensed electrical activity and/or other sensed signals, and provide the digitized signals to processing circuitry 50 for an initial detection analysis. Processing circuitry 50 may store in patient data 64 sensed signals and/or physiological parameters derived from such sensed signals by processing circuitry 50. For example, processing circuitry 50 may store cardiac EGM data encompassing a period of time (e.g., during which a cardiac episode may have occurred) in patient data 64. In some examples, the cardiac EGM data my include a sequence of values representing waves/waveforms (e.g., ECG-type waveforms) of a cardiac rhythm. It should be noted that the cardiac EGM data (e.g., for a typical ECG) may include a series of samples representing points on waves (e.g., a P-wave, Q-wave, R-wave, S-wave, T-wave, and/or U-wave), intervals (e.g., PR interval, QRS interval (also called QRS duration), QT interval, or RR interval), segments (e.g., PR segment, ST segment or TP segment), complex(es) (e.g., QRS complex), and/or other components. Processing circuitry 50 may apply a pattern recognition technique to interpret electrical activity vectors recorded in corresponding cardiac EGM data as one or more of the above components. In some examples, the waveform may indicate an initial detection of a cardiac event. Processing circuitry 50 may generate for output (e.g., via communication circuitry 54 to external device 12) data indicative of a particular cardiac event type as a classification of the detected cardiac event. In some examples, external device 12 may display the classification of the detected cardiac event.

IMD 10 may store specific threshold(s) (e.g., minimum/maximum or a range), aggressiveness levels, options, flags, and/or the like in arrhythmia criteria 67. Processing circuitry 50 may use arrhythmia criteria 67 may be used for detecting an occurrence of an arrhythmia in time-stamped patient information (e.g., a cardiac EGM segment).

Processing circuitry 50 and/or sensing circuitry 52 may read/write the patient data from/to storage device 56. Processing circuitry 50 and/or sensing circuitry 52 may cooperate to continuously record (e.g., monitor) the cardiac EGM data or ECG data, for example, as a graph of two-dimensional points and/or vectors. Processing circuitry 50 and/or communication circuitry 54 may upload the patient data via a communication channel (e.g., a wireless connection, such as Bluetooth®) to a remote device, such as external device 12 of FIG. 1.

Processing circuitry 50 and/or communication circuitry 54 may transmit patient data 64 via a communication channel to external device 12. In some examples, processing circuitry 50 and/or communication circuitry 54 may transmit patient data, sensed by or derived by a medical tool such as tool 6 of FIG. 1, to external device 12.

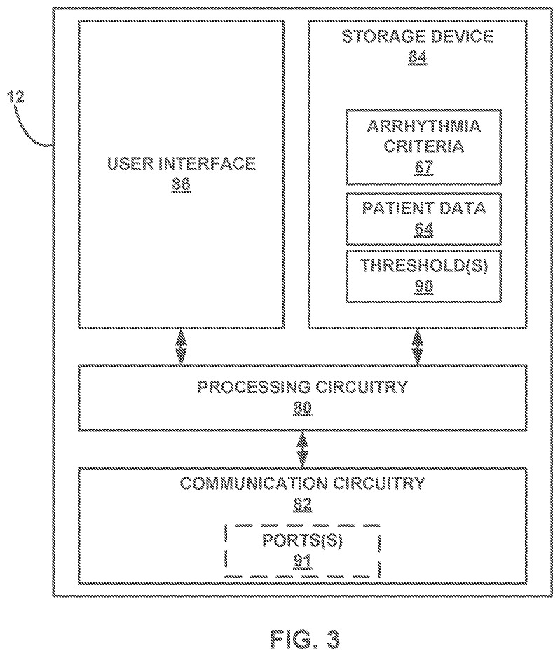

FIG. 3 is a block diagram illustrating an example configuration of components of external device 12. In the example of FIG. 3, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICS, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10 and/or tool 6. Under the control of processing circuitry 80, communication circuitry 82 may receive downlink telemetry from, as well as send uplink telemetry to, IMD 10, or another device, such as tool 6. In some examples, communication circuitry 82 may receive communications from and transmit communications to tool 6 via a wired connection between external device 12 and tool 6. Communication circuitry 82 may be configured to transmit or receive signals via inductive coupling, electromagnetic coupling, NFC, RF communication, Bluetooth, WiFi, or other proprietary or non-proprietary wireless communication schemes. Communication circuitry 82 may also be configured to communicate with devices, such as tool 6, via any of a variety of forms of wired and/or wireless communication and/or network protocols.

Communication circuitry 82 may include one or more port(s) 91. Port(s) 91 may be configured to communicatively couple external device 12 to tool 6 (and/or wearable device 20), via a wired or optical connection.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution. Storage device 84 may store threshold(s) 90 which may be used by processing circuitry 80 to determine an appropriate site for IMD 10 to be implanted or wearable device 20, to be located. For example, threshold(s) (90) may include an R-wave threshold, an impedance threshold, a signal strength threshold, a heart sound volume threshold, and/or a respiration sound volume threshold, to which sensor data of an R-wave amplitude, an impedance, a communication signal strength, a heart sound volume, and/or a respiration sound volume, respectively, may be compared.

Data exchanged between external device 12 and IMD 10 may include operational parameters. External device 12 may transmit data including computer readable instructions which, when implemented by IMD 10, may control IMD 10 to change one or more operational parameters and/or export collected data (e.g., patient data 64). For example, processing circuitry 80 may transmit an instruction to IMD 10 which requests IMD 10 to export collected data to external device 12. In turn, external device 12 may receive the collected data from IMD 10 and store the collected data in storage device 84 (e.g., in patient data 64). The data external device 12 receives from IMD 10 may include episode data (e.g., cardiac EGMs), sensor data, and other patient data described herein. Processing circuitry 80 may implement any of the techniques described herein to analyze data from IMD 10 to determine parameter values e.g., to determine whether the patient is experiencing a change in health e.g., based upon one or more criteria.

A user, such as a clinician or patient 4, may interact with external device 12 through user interface 86. User interface 86 may include a display (not shown), such as a liquid crystal display (LCD) or a light emitting diode (LED) display or other type of screen, with which processing circuitry 80 may present information related to IMD 10 and/or tool 6, e.g., sensor data corresponding to electrical activity in or around the heart (e.g., heart signals). In addition, user interface 86 may include an input mechanism configured to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

In some examples, processing circuitry 80 of external device 12 may monitor signals carrying patient information captured by way of clinician use of tool 6, for example, after implantation of IMD 10, while actively implanting IMD 10, or testing a location for implanting IMD 10. The patient information may be in the form of the above-mentioned sensor data. Similar to how external device 12 may interrogate IMD 10 to receive signals (e.g., heart signals), external device 12 may interrogate tool 6 to receive signals via communication circuitry 82. In this manner, a processor within tool 6 may generate data storing the signals. Communication circuitry of tool 6 may be operative to transmit the stored data to external device 12 for storage within local memory. External device 12 may retrieve the stored data from the local memory to run a test to determine a location's suitability as an implantation site (and/or attachment site). Details regarding examples of this test are provided further below, for example, in the description of FIGS. 5A-5C.

Processing circuitry 80 of external device 12 may also generate and store marker codes indicative of different cardiac episodes and/or other health events. This may be done as part of performing an example test, such as to determine an expected accuracy in health event detection should IMD 10 be implanted at a particular location. In some examples, sensing circuitry of tool 6 and/or IMD 10 may be configured to detect the marker codes prior to the communication circuitry transmitting them to external device 12. Communication circuitry 82 may receive the marker codes at external device 12.

In one example, external device 12 and tool 6 may include ports configured as interfaces which may be interconnected by wire or optical cables to each other to enable transmissions of data, including sensor data generated from signals captured by tool 6. In other examples, external device 12 and tool 6 (e.g., via a leadless embodiment of IMD 10) may be BLE, or Bluetooth-enabled, or other wireless communication protocol-enabled devices, and therefore, be capable of communicating the sensor data to each other via a radio access technology (RAT). It should be noted that these device(s) may leverage any type of wireless, wired, optical, or network connection for completing the transmission of the sensor data. IMD 10 may run software code (e.g., of one or more communication protocols) to delineate the sensor data in a message for transmission (e.g., via a wireless connection) as packetized data.

External device 12 may store specific threshold(s) (e.g., minimum/maximum or a range), aggressiveness levels, options, flags, and/or the like in arrhythmia criteria 67. Arrhythmia criteria 67 may be used in an algorithm for detecting an occurrence of an arrhythmia in time-stamped patient information (e.g., a cardiac EGM segment). One example use of the sensor data to improve performance may involve adjusting one or more of the above-mentioned thresholds, aggressiveness levels, options, flags, and/or the like based on various measurements corresponding to cardiac activity, for example, in the form of electrical signals. The settings/parameters may have been pre-determined by the clinician, but that pre-determination may have been made on inaccurate data or insufficient data. Thus, the sensor data provided by tool 6 may be used to update the pre-determined settings/parameters. As an option, a portion of the sensor data may correspond to different patient monitoring/detection functionality of an alternative cardiac monitoring device, allowing a comparison of their respective compare efficacies.

There are a number of advantages from providing the implanting clinician with insights into the implant quality during (including both finding an appropriate location for IMD 10 and inserting or implanting IMD 10) or after the implantation procedure. Enabling the implanting clinician to test and evaluate the implant reduces the likelihood of faulty/suboptimal performance or even complete malfunction. As examples, low amplitude R-waves may result in suboptimal sensing performance, poor BLE signal strength may result in connection drop-outs, among others. Adequate R-wave amplitude may be relied upon by detection algorithms. Improving R-wave amplitude (e.g., via appropriate device location) reduces ‘false-positives’ and ‘false-negatives’ and may prevent the need to reposition or explant medical devices that are not able to collect such diagnostic data. Connection drop-outs due to poor BLE signal strength can be costly from a battery longevity and a ‘time to transmit’ standpoint, as it may require sending duplicate data using an energy-intensive RF module. There may be an increased risk of connection drop-out when the patient is ambulatory (due to environmental considerations, proximity to the monitor, etc.). Ensuring optimal signal strength (ex: RSSI) at the time of connection may minimize or reduce this risk.

In some examples, a signal strength threshold may be a range. If, after implantation of IMD 10, a sensed BLE signal strength is outside of the threshold range (e.g., higher or lower than the threshold range), processing circuitry 80 may provide, via communication circuitry 82, an instruction to IMD 10 to adjust receiver strength and/or transmitter power in an attempt to optimize battery longevity versus wireless communication performance.

As will be discussed in greater detail below with respect to FIG. 4, one or more remote computing devices may interact with IMD 10 in a manner similar to external device 12, e.g., to program IMD 10 and/or retrieve data from tool 6, via a network. For instance, a remote computing service, which may be similar to that provided by the Medtronic CareLink® Network, may communicate with IMD 10 directly over a network connection and/or indirectly through external device 12.

FIG. 4 is a block diagram illustrating an example system that includes one or more computing devices, including monitoring device 90 and programmer device 91, communicatively coupled to medical tool 6.

Monitoring device 90 and programmer device 91 represent two examples of a number of different examples of an external computing device configured to receive sensor data generated by tool 6. In some examples, monitoring device 90 and/or programmer device 91 may represent examples of external device 12. Other example external computing devices, such as a server 94, and one or more other computing devices 99A-99N (collectively, “computing devices 99”) may be coupled to tool 6, IMD 10, monitoring device 90 and/or programmer device 91 via network 92, in accordance with one or more techniques described herein. In some examples, at least one of the other example devices may be configured to receive the same sensor data. In this example, tool 6 may use communication circuitry (e.g., communication circuitry) to communicate with any of the above-mentioned external computing devices via a wired and/or wireless connection. In the example of FIG. 5, the other computing devices 99 are interconnected and may communicate with each other through network 92.

In some cases, server 94 may be configured to provide a secure storage site for data that has been collected from tool 6, IMD 10, monitoring device 90, and/or programmer device 91. In some cases, server 94 may assemble data in web pages or other documents for viewing by trained professionals, such as clinicians, via computing devices 99. One or more aspects of the illustrated system of FIG. 4 may be implemented with general network technology and functionality, which may be similar to that provided by the Medtronic CareLink® Network.

In the example illustrated by FIG. 4, server 94 includes a storage device 96, e.g., to store data retrieved from tool 6, IMD 10, monitoring device 90, and/or programmer device 91, and processing circuitry 98. Although not illustrated in FIG. 4, computing devices 99 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuity of computing devices 99 may implement any of the techniques described herein to analyze sensor data generated by tool 6 to evaluate an implant quality corresponding to IMD 10.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

In some examples, one or more of computing devices 99 may be a smartphone, tablet computer, or other smart device located with a clinician, by which the clinician may program, receive alerts from, and/or interrogate IMD 10 and/or tool 6. For example, the clinician may access various patient data including instrument measurements, sensor data, physiological parameters of IMD 10, electrocardiogram, and/or indications and/or indications of patient health collected by IMD 10 through a computing device 99, such as when patient 4 is in in between clinician visits, to check on a status of a medical condition.

FIGS. 5A-5C are illustrations of medical tools 100A-100C for use in an implantation procedure of IMD 10 of FIGS. 1-2, in accordance with one or more examples of the present disclosure. Each illustration depicts a different example of medical tool 6 of FIG. 1 where each medical tool 100 is configured with a pair of electrodes 110A and 110B formed on or in a housing 114 and to sense electrical activity associated with a body (or portion thereof) of patient 4. To enable a meaningful evaluation of a body part for receiving an implantation of a medical device, the pair of electrodes 110A and 110B may be configured on medical tools 100 to correspond with electrodes (e.g., electrodes 16A and 16B) of a medical device (e.g., IMD 10) to be implanted into patient 4. For example, to correspond to electrodes 16A and 16B, electrodes 110A and 110 B may be configured to sense one or more of the same physiological parameters or signals as IMD 10 (or an external device), the spacing between electrodes 110A and 110B may be the same or similar the spacing between electrodes 16A and 16B, electrodes 16A and 16B may have a same or similar impedance as electrodes 110A and 110B, or the like.

In each of the illustrations, the example medical tool 100 is depicted as having been manufactured/adapted for partial insertion into a body of the patient 4 and to test a location corresponding to the candidate implantation site for IMD 10. As such, the housing of medical tool 100A may be composed of any material capable of being adapted to a rigid body for the partial insertion. Electrodes 110 may be example sensors configured for placement of in or around a thoracic cavity of the body of patient 4. Similar to other sensor types, electrodes 110 may form on or in the housing of medical tool 100A such that electrodes 110 have the same or similar configuration as corresponding electrodes of IMD 10.

Respective medical tools 100 depicted in FIGS. 5A-5C further include (in some form) communication circuitry configured to transmit, to a computing device (e.g., external device 12), sensor data generated from signals captured or sensed by electrodes 110 and/or other one or more sensors. The computing device is described herein as being configured to run a test on the sensor data to identify an (appropriate) implantation site for the medical device implantation.

The above-mentioned communication circuitry generally comprises circuitry or circuitries, including circuitry known as communication circuitry, and any suitable firmware, software, other hardware, or any combination thereof for communicating with another device, such as external device 12. Under the control of processing circuitry, the communication circuitry may receive downlink telemetry from and send uplink telemetry to external device 12 with the aid of an antenna, which may be internal and/or external. The processing circuitry may a separate hardware element from the communication circuitry and therefore, allocatable to other hardware. In another example, the processing circuitry may be a component of (e.g., a circuit within) the communication circuitry and therefore, a dedicated processor to the communication circuitry. The processing circuitry may provide data to be uplinked to external device 12 in the form of signals for the communication circuitry, e.g., via an address/data bus, to relay (e.g., receive and then, transmit) to external device 12. In some examples, the communication circuitry may provide captured signals and/or sensor data generated from such signals to the processing circuitry via a multiplexer. As another alternative, separate (e.g., dedicated) logic circuitry within external device 12 may generate and store sensor data including the marker codes indicative of different cardiac episodes and/or other health events.

In the example of FIG. 5A, medical tool 100A is depicted with a pair of ports 120A and 120B (hereinafter referred to as “ports 120”) corresponding to the pair of electrodes 110A and 110B, respectively, and configured to route, via a wired or optical connection, signals indicative of electrical activity of a location within a patient, for example, in or around a thoracic cavity of the patient's heart, to a computing device (e.g., external device 12). Ports 120 may be located on or in handle 115 of medical tool 100A. One or both ports 120 may be adapted to mate with or affix to compatible interconnect of any computing device. For instance, one or both of ports 120 may be communicatively coupled to the computing device, such as monitoring device 90, by mating with a compatible interconnect on that computing device and then, transporting (e.g., raw) sensor data to logic circuitry of that computing device. One example of monitoring device 90 may be a cardiac EGM/ECG monitor that is instrumented to receive/capture signals of electrical activity of a candidate implantation site and/or then, generate sensor data comprising measurements indicating electrical activity.

The same monitoring device or another external device, a computing device, may store the sensor data in memory and proceed to evaluate the location from which the electrical activity is sensed as to whether the location an appropriate implantation site for a medical device, such as IMD 10. The present disclosure further specifies that the type of medical device may be any IMD.

An example embodiment of port(s) 120 may comprise an insulating cover over a conductive material configured to couple with one or more corresponding connectors of the computing device. Ports 120 may include a portion of the communication circuitry that, in conjunction with a communication system (e.g., a bus), form connection/contact points through which respective electrodes 110 (e.g., a source) propagate electrical activity (e.g., as carrier signals) to a computing device, such as external device 12 (e.g., a sink). The external device may include a programmer device for the medical device among other examples. Electrodes 110 may be formed from any of stainless steel, titanium, platinum, iridium, or alloys thereof. In addition, electrodes may be coated with a material such as titanium nitride or fractal titanium nitride, although other suitable materials and coatings for such electrodes may be used.

In the example of FIG. 5A, medical tool 100A may be an implanter device and may include recess 112 for receiving IMD 10. Medical tool 100A may include a mechanism (not shown) for implanting IMD 10 into patient 4. Any example medical tools, such as tool 6, described herein, may be include recess 112 and may be an implanter device. More information regarding such an implanter device may be found in U.S. Pat. No. 11,311,213 issued on Apr. 26, 2022, the entirety of which is incorporated by reference.

In the example of FIG. 5B, medical tool 100B is depicted with a pair of connectors 130A and 130B (collectively “connectors 130”) that each correspond to a respective one of the pair of electrodes 110A and 110B and may feed sensor data directly to another device. For example, connectors 130 may be configured to connect with connector(s) 66 of IMD 10 (FIG. 2) for the purpose of transmitting sensor data from medical tool 100B to IMD 10. Alternatively, or additionally, connectors 130 may be configured to connect with connectors of wearable device 20 (not shown for simplicity purposes). In turn, communication circuitry 54 of IMD 10 may proceed to transmit the sensor data to external device 12 upon receiving the sensor data from medical tool 100B.

One or both of connectors 130 may be any mechanical-electrical component capable of providing an electrical contact for reliable connectivity/communication; some examples of connectors 130 include mechanical-electrical components knowns as compression connectors or compression contacts. In general, each connector 130 is composed of a conductive material (e.g., a wire) and is configured to couple with one or more corresponding (e.g., compatible) interfaces of the above other device, including the medical device for the purposes of achieving a successful implantation.

Each connector 130 is manufactured with a periphery surface configured to establish a coupling with a compatible interface. One example of such an interface may include electrical-mechanical hardware through which connector(s) 130 may be mounted to a printed circuit board (PCB) or mated with logic circuitry on that board. Mounting blocks, as one example of the above electrical-mechanical hardware, may be coupled with compression connectors to form a compression connection.

Each electrode 110 may have a same size, inductance, resistance to shock, vibration, and/or thermal cycling as a corresponding electrode on IMD 10. When placed onto the housing, the electrodes 110 of medical tool 100B may be positioned on outer periphery. Connectors 130 may be formed in a position in a gap defined by electrodes 110 and handle 115. The distal end of medical tool 100B is configured to be inserted into patient 4. Connectors 130 are adapted to electrically contact/connect one or more interfaces to one or more hardware components (e.g., circuities such as switching circuitry). To produce its rigid body, the housing may be formed from plastic, titanium, or any other suitable material (e.g., a biocompatible material).

Interfaces such as the mounting blocks may be used for interconnecting (e.g., solderless) connectors to IMD 10. An example connector 130 may be a compression connecter that provides a mechanical and electrical contact whose construction includes tightly-wound metallic wire formed into a cylindrical shape. The example connector 130 may be installed into an insulator or a housing. The contact may extend to both sides of the insulator, creating an electrical connection capable of handling electrical signals.

In the example of FIG. 5C, medical tool 100C is configured with communication circuitry 140 for transmitting, via a wireless connection, sensor data indicative of electrical activity sensed by the pair of electrodes 110. One example of the wireless connection may be a Bluetooth® connection. Similar to the external device and the medical device, medical tool 100C may establish a communication channel (e.g., uplink telemetry) for receiving/sending data. Using the communication channel, external device 12 may test the sensor data provided by medical tool 100C and evaluate the sensed location as an implantation site.

While three separate examples of tool 6 have been set forth in FIGS. 5A-5C, in some examples, tool 6 may include any combination of features set forth in FIGS. 5A-5C. For example, tool 6 may include more than one feature for communicating sensor data from tool 6 to external device 12 (e.g., any combination of ports 120, connectors 130, and/or communication circuitry 140).

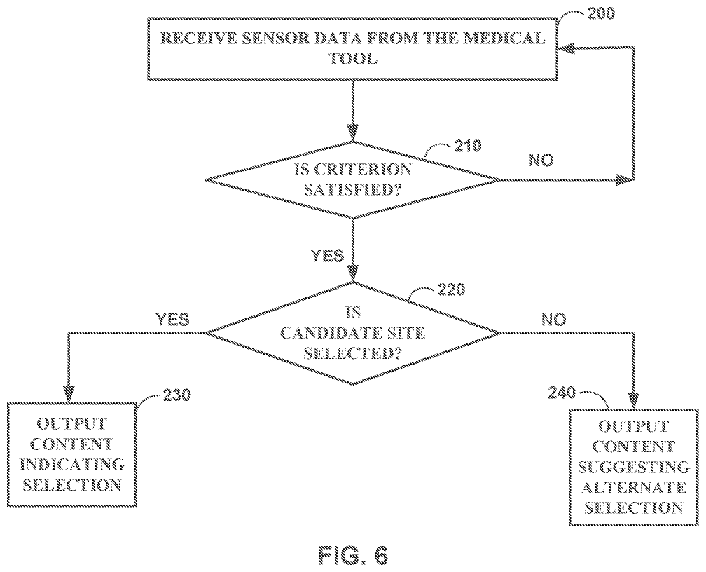

FIG. 6 is a flow diagram illustrating an example operation for performing a perioperative evaluation in support of a medical device implantation, in accordance with one or more examples of the present disclosure. In some examples, the example operation may be implemented for a cardiac monitoring device, such as IMD 10 of FIG. 1, and enabled by an implant delivery tool, such as tool 6 of FIG. 1, as part or in preparation of an implantation procedure for the cardiac monitoring device.

In the example of FIG. 6, processing circuitry 80 of external device 12, being operated by a clinician, may initiate a test to find a suitable or best possible location within a body of patient 4 for the medical device implantation. For example, the test may be initiated on tool 6 or external device 12 based on clinician or user input. This test generally involves processing sensor data sensed by the medical tool according to one or more criterion and considering the location corresponding to the sensor data as a candidate implantation site. As described herein, having sensor(s) which are configured the same as or similar to sensor(s) of the medical device to be implanted (e.g., IMD 10) into patient 4 provides perioperative insight regarding the candidate implantation site, generally, in terms of performance, should the medical device actually be implanted into the corresponding location. The clinician may insert a portion of tool 6 into patient 4 and may position tool 6 such that sensor(s) of tool 6 may be located at, or proximate to, the corresponding intended location of sensor(s) of IMD 10 at the candidate implantation site. This positioning of tool 6 may include both an insertion site and an insertion angle, for example, relative to the body of patient 4.

In the example operation of FIG. 6, processing circuitry 80 of external device 12 may receive sensor data captured by one or more sensors of a medical tool (220). For example, in the case where IMD 10 is an example medical device to be implanted in patient 4, one example candidate implantation site may correspond to a location in or around a thoracic cavity. Placing sensor(s) of tool 6 near or in contact with the corresponding location allows tool 6 to sense or capture signals similar to those which would be expected to be captured by IMD 10 if IMD 10 is implanted at the candidate implantation site, for example, signals indicative of some cardiac activity. The one or more sensors of tool 6 may have a corresponding configuration to one or more sensors of IMD 10 to be implanted in a body of patient 4.

Tool 6 may, via electrodes 110 (see FIGS. 5A-5C), may capture signals indicative of electrical activity, for example, of heart 8 or patient 4. In some examples, sensing circuitry in IMD 10, external device 12, or in tool 6 may generate the sensor data from these captured signals. As mentioned above, sensor data may include sensor signals, and/or physiological parameters and/or other parameters which may be derived from the sensor signals.

Processing circuitry 80 may determine whether at least one criterion is satisfied (210). For example, processing circuitry 80 may execute the test to evaluate the candidate implantation site based on the sensor data. There are a number of applicable metrics for evaluating the candidate implantation site, such as measuring/analyzing certain parameters from the sensor data. Processing circuitry 80 may, therefore conduct one or more tests to determine whether the candidate implantation site is appropriate (or more appropriate than other sites) for implantation of IMD 10. For example, processing circuitry 80 may compare sensor data to one or more of threshold(s) 90. For example, processing circuitry 80 may determine whether an amplitude of a sensed R-wave meets a predetermined R-wave threshold. For example, the amplitude of the sensed R-wave may meet the R-wave threshold if it is greater than (or greater than or equal to) the R-wave threshold. For example, the R-wave threshold may be 150 mV, 175 mV, 200 mV, 225 mV, or any other predetermined amplitude value. In one example, the R-wave threshold is 200 mV. Alternatively, or additionally, processing circuitry 80 may compare sensor data to other thresholds, such as an impedance threshold, a signal strength threshold, and/or the like. For example, processing circuitry 80 may compare a BLE signal strength of a BLE beacon from an external device sensed by tool 6 to a predetermined BLE threshold to determine (or as part of determining) whether the candidate implantation site is appropriate for implantation.

For example, if the amplitude of the sensed R-wave meets the R-wave threshold, the at least one criterion may be satisfied. Responsive to determining that the at least one criterion is satisfied (the “YES” path from box 250), processing circuitry 80 of external device 12 may determine whether to select the candidate implantation site for the medical device implantation (220). In some examples, a clinician may determine to select the candidate implantation site for IMD 10 based, at least in part, on the at least one criterion being satisfied. In other examples, processing circuitry 80 may determine whether to select the candidate implantation site for IMD 10. In such cases, the clinician may override the determination of processing circuitry 80 and implant IMD 10 at the candidate implantation site. It should be noted that in some examples, processing circuitry 80 may evaluate a candidate implantation site by comparing test results with a plurality of criteria. In some examples, satisfying a single criterion of the plurality of criteria may not result in the selection of a candidate implantation site. In some examples, processing circuitry 80 may select the candidate implantation site if a predetermined number of criterion (e.g., every one of the criteria, a majority of the criteria, or another predetermined number of criteria) is be satisfied.

In one example, processing circuitry 80 may determine that the candidate implantation site is more appropriate (e.g., a better or best fit) for receiving IMD 10 as an implant than other potential candidate implantation sites. This determination may be based (at least in part) on sensor data provided by tool 6. This sensor data may be predicative of an implant quality at the candidate implantation site, for example, due to resembling the same sensor data that IMD 10 would sense at the same location/site. Accordingly, processing circuitry 80 may generate an indication, which may include text that is descriptive of the above determination and suggestive of the candidate implantation site.

There are tests to measure signals (e.g., heart signals) in terms of quality and/or other metrics. In some examples, processing circuitry 80 may execute these tests to select a location associated with highest signal accuracy for the implant. A typical test as described herein generally involves mechanically/physically positioning tool 6 in patient 4 to sense electrical activity of interest and evaluate an implant quality of a location being sensed (which may be defined in terms of accuracy among other attributes). Searching for a location where a clinician can expect to receive at least a minimum level of accuracy, external device 12 may repeat this test for each of a set of candidate implantation sites and (perhaps) find a location where it would be possible for IMD 10 to receive more informative or higher quality signals than other sites. This allows external device 12, on behalf of the clinician, to identify an implantation site that is expected to provide the most benefit to patient 4 (assuming such a site actually exists). Such a test may allow the clinician to find the location where the implant would be (e.g., most) effective, assuming that having more accurate sensor data improves performance in the medical device being implanted.

Based on a determination to select the candidate implantation site for the implantation (the “YES” path from box 220), processing circuitry 80 may generate, for output via a display device, an indication including content indicating the selection of the selected candidate implantation site (230), which, in some examples, may be followed by a description of the test results.

In some examples, processing circuitry 80 may determine to not select the candidate implementation site for the implementation. For example, processing circuitry 80 may compare test results (or sensed signals) from another site to test results (or sensed signals) from the candidate implementation site and determine that the sensed signals captured at the other site are more informative or of higher quality than those of the candidate implantation site. Based on a determination not to select the candidate implantation site for the implantation (the “NO” path from box 220), processing circuitry 80 may generate and output an indication including content suggesting an alternate location to select as the implantation site (240). For example, external device 12 may keep track of various candidate implantation sites that have been tested, and store information relating to test results in storage device 84 (e.g., in patient data 64). Processing circuitry 80 may then determine a suggested alternate location, such as one not previously tested. In some examples, processing circuitry 80 may compare test results for previously tested candidate implantation sites and determine a suggested alternate location by selecting a location closer to a candidate implantation site having better test results and further from a candidate implantation site having worse test results. For example, better test results may include satisfying more criteria, or being closer to satisfying one or more criteria than worse test results. For example, the content suggesting an alternate location may inform the clinician that the clinician my attempt testing at one or more alternate locations. The content suggesting an alternate location may or may not indicate a suggested alternate location. For example, the indication may indicate to try one or more specific locations or may not indicate to try any specific location. In some examples, external device 12 may include a camera sensor which may be configured to capture an image of IMD 10. Processing circuitry 80 may determine a size of IMD 10 based on the captured image. Processing circuitry 80 may utilize the size of IMD 10 when determining a suggested alternate location. For example, a smaller IMD may be better placed closer to heart 8, than a larger IMD.

One example test provides a perioperative implant quality evaluation for ICMs in general and any test results may be used to improve the implantation procedure, for instance, by being at least informative to the clinician as to a preferred or acceptable implantation site. Using tool 6, the example test may involve the clinician positioning one or more electrodes of tool 6 in contact with or near the candidate implantation site. In the example, where the device is not an IMD, but is a wearable device, the clinician may position one or more electrodes of tool 6 in contact with skin of patient 4 at or near a candidate site for the wearable device. Proper positioning may require placing the one or more electrodes at a location that is likely to be the same as or proximal to a location from which one or more corresponding electrodes of IMD 10 (or wearable device 20) is to sense electrical signals. Example test results may enable processing circuitry 80 (e.g., and the clinician) to determine and suggest a location for implanting IMD 10 (or positioning wearable device 20). Based on the example test results, processing circuitry 80 may output an indication including content suggesting or directing the clinician to implanting IMD 10 (or positioning wearable device 20) on or around a suitable location for detecting cardiac events, including specific arrhythmia types.

An alternative, or addition, to the above test may be a perioperative evaluation for providing therapy. Consider an example where IMD 10, equipped with appropriate therapy delivery circuitry, is to be implanted into patient 4. In such an example, processing circuitry 80 may evaluate a candidate implantation site for therapy delivery in addition to, or instead of, cardiac monitoring/health event detection. Similar to the former example test, this test may involve the clinician positioning one or more electrodes of tool 6 in contact with or near the candidate implantation site where proper positioning may require placing the one or more electrodes in contact with a location that is likely to be the same as or proximate to a location to contact one or more corresponding electrodes of IMD 10; however, the alternative test employs tool 6 to evaluate the location for suitability in delivering therapy (e.g., via the therapy delivery circuitry). One example therapy that IMD 10 may deliver includes cardioversion or defibrillation shock with the aid of an output circuit that determines whether a monophasic or biphasic pulse is delivered, whether housing electrode serves as cathode or anode, and which electrodes are involved in delivery of the cardioversion or defibrillation pulses. Such functionality may be provided by one or more switches or a switching circuitry of therapy delivery circuitry of the implantable device.

The order and flow of the operation illustrated in FIG. 6 are examples. In some examples according to this disclosure, any number of thresholds may be considered. Further, in some examples, processing circuitry may perform or not perform certain steps of the method of FIG. 6, or any of the techniques described herein, as directed by a user, e.g., via external device 12, or computing devices 99.

In some examples, tool 6 includes housing 114 for one or more sensors (e.g., electrodes 110), the housing including a rigid body configured to be at least partially inserted into a body of patient 4, the one or more sensors having a corresponding configuration to one or more sensors (e.g., electrodes 16) of a medical device (e.g., IMD 10) to be implanted in the body of the patient. Tool 6 includes communication circuitry (e.g., ports 120, connectors 130, and/or communication circuitry 140) configured to transmit, to a computing device (e.g., external device 12), sensor data generated from signals captured by the one or more sensors. In some examples, the computing device is configured to run a test on the sensor data to identify an implantation site from at least one candidate implantation site for the medical device.

In some examples, the identified implantation site is in a thoracic cavity of the body of the patient. In some examples, the medical device includes a cardiac monitoring device. In some examples, the cardiac monitoring device includes an insertable cardiac monitor.

In some examples, the communication circuitry includes one or more connectors (e.g., connectors 130) including a conductive material and configured to couple with one or more corresponding connectors of the medical device. In some examples, the communication circuitry is configured to transmit the sensor data to the computing device via the one or more connectors and communication circuitry of the medical device.

In some examples, the communication circuitry includes at least one port (ports 120) configured to couple with at least one corresponding port of the computing device via at least one wire or optical fiber, and wherein the communication circuitry is configured to transmit the sensor data to the computing device via the at least one port.

In some examples, the communication circuitry (e.g., communication circuitry 140) is configured to couple to the computing device via a wireless connection, and wherein the communication circuitry is configured to communicate the sensor data via the wireless connection to the computing device.

In some examples, the medical tool (e.g., medical tool 100A) is configured to insert the medical device into the body of the patient. In some examples, the corresponding configuration includes at least one of a same sensor type, a same electrode spacing, or a same electrode impedance.

In some examples, the test includes determining whether at least one aspect of the sensor data satisfies at least one criterion. In some examples, the at least one criterion includes at least one of an R-wave amplitude threshold, an impedance threshold, or a wireless signal strength threshold. In some examples, satisfying at least one criterion includes satisfying more than one criterion.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as clinician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

The following examples are a non-limiting list of clauses in accordance with one or more techniques of this disclosure.
Example 1. A medical tool comprising: a housing for one or more sensors, the housing comprising a rigid body configured to be at least partially inserted into a body of a patient, the one or more sensors having a corresponding configuration to one or more sensors of a medical device to be implanted in the body of the patient; and communication circuitry configured to transmit, to a computing device, sensor data generated from signals captured by the one or more sensors, wherein the computing device is configured to run a test on the sensor data to identify an implantation site from at least one candidate implantation site for the medical device.
Example 2. The medical tool of Example 1, wherein the identified implantation site is in a thoracic cavity of the body of the patient.
Example 3. The medical tool of Example 1 or Example 2, wherein the medical device comprises a cardiac monitoring device.
Example 4. The medical tool of Example 3, wherein the cardiac monitoring device comprises an insertable cardiac monitor.
Example 5. The medical tool of any of Examples 1-4, wherein the communication circuitry comprises one or more connectors comprised of a conductive material and configured to couple with one or more corresponding connectors of the medical device.
Example 6. The medical tool of Example 5, wherein the communication circuitry is configured to transmit the sensor data to the computing device via the one or more connectors and communication circuitry of the medical device.
Example 7. The medical tool of any of Examples 1-6, wherein the communication circuitry comprises at least one port configured to couple with at least one corresponding port of the computing device via at least one wire or optical fiber, and wherein the communication circuitry is configured to transmit the sensor data to the computing device via the at least one port.
Example 8. The medical tool of any of Examples 1-7, wherein the communication circuitry is configured to couple to the computing device via a wireless connection, and wherein the communication circuitry is configured to communicate the sensor data via the wireless connection to the computing device.
Example 9. The medical tool of any of Examples 1-8, wherein the medical tool is configured to insert the medical device into the body of the patient.
Example 10. The medical tool of any of Examples 1-9, wherein the corresponding configuration comprises at least one of a same sensor type, a same electrode spacing, or a same electrode impedance.
Example 11. A medical system comprising: a medical tool, the medical tool comprising a housing for one or more sensors, the housing comprising a rigid body configured to be at least partially inserted into a body of a patient, the one or more sensors having a configuration corresponding to one or more sensors of a medical device to be implanted in the body of the patient, and communication circuitry configured to transmit, to a computing device, sensor data generated from signals captured by the one or more sensors; and the computing device, wherein the computing device is configured to run a test on the sensor data to identify an implantation site from at least one candidate implantation site for the medical device.
Example 12. The medical system of Example 11, wherein the identified implantation site is in a thoracic cavity of the body of the patient.
Example 13. The medical system of Example 11 or Example 12, wherein the medical device comprises a cardiac monitoring device.
Example 14. The medical system of any of Examples 11-13, wherein the communication circuitry comprises one or more connectors comprised of a conductive material and configured to couple with one or more corresponding connectors of the medical device, and wherein the communication circuitry is configured to transmit the sensor data to the computing device via the one or more connectors and communication circuitry of the medical device.
Example 15. The medical system of any of Examples 11-14, wherein the communication circuitry comprises at least one port configured to couple with at least one corresponding port of the computing device via at least one wire or optical fiber, and wherein the communication circuitry is configured to transmit the sensor data to the computing device via the at least one port.
Example 16. The medical system of any of Examples 11-15, wherein the communication circuitry is configured to couple to the computing device via a wireless connection, and wherein the communication circuitry is configured to communicate the sensor data via the wireless connection to the computing device.
Example 17. The medical system of any of Examples 11-16, wherein the test comprises determining whether at least one aspect of the sensor data satisfies at least one criterion.
Example 18. The medical system of Example 17, wherein the at least one criterion comprises at least one of an R-wave amplitude threshold, an impedance threshold, a wireless signal strength threshold, a heart sound volume threshold, or a respiration sound volume threshold.
Example 19. The medical system of Example 17, wherein satisfying at least one criterion comprises satisfying more than one criterion.
Example 20. A method comprising: receiving, via communication circuitry, sensor data generated from signals captured by one or more sensors of a medical tool, the one or more sensors having a corresponding configuration to one or more sensors of a medical device to be implanted in a body of a patient; determining whether at least one criterion is satisfied; and outputting an indication of whether the at least one criterion is satisfied.

1. A medical tool comprising:
a housing for one or more sensors, the housing comprising a rigid body configured to be at least partially inserted into a body of a patient, the one or more sensors having a corresponding configuration to one or more sensors of a medical device to be implanted in the body of the patient; and
communication circuitry configured to transmit, to a computing device, sensor data generated from signals captured by the one or more sensors, wherein the computing device is configured to run a test on the sensor data to identify an implantation site from at least one candidate implantation site for the medical device.

2. The medical tool of claim 1, wherein the identified implantation site is in a thoracic cavity of the body of the patient.

3. The medical tool of claim 1, wherein the medical device comprises a cardiac monitoring device.

4. The medical tool of claim 3, wherein the cardiac monitoring device comprises an insertable cardiac monitor.

5. The medical tool of claim 1, wherein the communication circuitry comprises one or more connectors comprised of a conductive material and configured to couple with one or more corresponding connectors of the medical device.

6. The medical tool of claim 5, wherein the communication circuitry is configured to transmit the sensor data to the computing device via the one or more connectors and communication circuitry of the medical device.

7. The medical tool of claim 1, wherein the communication circuitry comprises at least one port configured to couple with at least one corresponding port of the computing device via at least one wire or optical fiber, and wherein the communication circuitry is configured to transmit the sensor data to the computing device via the at least one port.

8. The medical tool of claim 1, wherein the communication circuitry is configured to couple to the computing device via a wireless connection, and wherein the communication circuitry is configured to communicate the sensor data via the wireless connection to the computing device.

9. The medical tool of claim 1, wherein the medical tool is configured to insert the medical device into the body of the patient.

10. The medical tool of claim 1, wherein the corresponding configuration comprises at least one of a same sensor type, a same electrode spacing, or a same electrode impedance.

11. A medical system comprising:
a medical tool, the medical tool comprising a housing for one or more sensors, the housing comprising a rigid body configured to be at least partially inserted into a body of a patient, the one or more sensors having a configuration corresponding to one or more sensors of a medical device to be implanted in the body of the patient, and communication circuitry configured to transmit, to a computing device, sensor data generated from signals captured by the one or more sensors; and
the computing device, wherein the computing device is configured to run a test on the sensor data to identify an implantation site from at least one candidate implantation site for the medical device.

12. The medical system of claim 11, wherein the identified implantation site is in a thoracic cavity of the body of the patient.

13. The medical system of claim 11, wherein the medical device comprises a cardiac monitoring device.

14. The medical system of claim 11, wherein the communication circuitry comprises one or more connectors comprised of a conductive material and configured to couple with one or more corresponding connectors of the medical device, and wherein the communication circuitry is configured to transmit the sensor data to the computing device via the one or more connectors and communication circuitry of the medical device.

15. A method comprising:
receiving, via communication circuitry, sensor data generated from signals captured by one or more sensors of a medical tool, the one or more sensors having a corresponding configuration to one or more sensors of a medical device to be implanted in a body of a patient;
determining whether at least one criterion is satisfied; and
outputting an indication of whether the at least one criterion is satisfied.

16. The medical system of claim 11, wherein the communication circuitry comprises at least one port configured to couple with at least one corresponding port of the computing device via at least one wire or optical fiber, and wherein the communication circuitry is configured to transmit the sensor data to the computing device via the at least one port.

17. The medical system of claim 11, wherein the communication circuitry is configured to couple to the computing device via a wireless connection, and wherein the communication circuitry is configured to communicate the sensor data via the wireless connection to the computing device.

18. The medical system of claim 11, wherein the test comprises determining whether at least one aspect of the sensor data satisfies at least one criterion.

19. The medical system of claim 18, wherein the at least one criterion comprises at least one of an R-wave amplitude threshold, an impedance threshold, a wireless signal strength threshold, a heart sound volume threshold, or a respiration sound volume threshold.

20. The medical system of claim 18, wherein satisfying the at least one criterion comprises satisfying more than one criterion.
